(19) United States
(12) Patent Application Publication
(10) Pub. No.: US 2008/0199401 A1
(43) Pub. Date: Aug. 21, 2008

(54) Method for Dissolution of Gases with Short-Lived Physical Properties in a Liquid

(76) Inventors: Peter Blumler, Bielefeld (DE); Horst-Dieter Lemke, Obernburg (DE); Detlef Krieter, Wurzburg (DE); Hans-Wolfgang Spiess, Mainz (DE); Frank Wiese, Wuppertal (DE); Paul-Philipp Zanker, Mainz (DE)

Correspondence Address: HAMMER & ASSOCIATES, P.C., 3125 SPRINGBANK LANE, SUITE G, CHARLOTTE, NC, 28226, US

(21) Appl. No.: 11/916,662

(22) PCT Filed: Jun. 6, 2006
(86) PCT No.: PCT/EP2006/005349

§ 371 Date: Mar. 6, 2008

(30) Foreign Application Priority Data
Jun. 9, 2005 (DE) 10 2005 026 604.5

(51) Int. Cl. A61K 49/06

(52) U.S. Cl. 424/9.3

(57) ABSTRACT

The invention relates to a process for dissolving a gas with short-lived physical properties in a liquid, comprising the steps of providing the gas with short-lived physical properties, providing the liquid and bringing the gas with short-lived with short-lived physical properties is fed into the liquid via a semi-permeable membrane that is permeable to gas.

The invention relates to a process for dissolving a gas with short-lived physical properties in a liquid.

Gases with short-lived physical properties are, for example, radioactive gases or hyperpolarised gases. Radioactive gases are gases containing a so-called radionuclide or radioisotope. Such gases are employed among other things in positron emission tomography (PET).

Nuclear magnetic resonance spectroscopy (NMR) and imaging methods based on magnetic resonance, such as magnetic resonance tomography (MRT), employ hyperpolarised gases alongside other substances as contrast media. For certain applications improved images or an improved signal-to-noise ratio can be obtained with the above-mentioned magnetic resonance-based methods by using hyperpolarised gases as signal generators.

The use of hyperpolarised noble gases for NMR or MRT techniques is described, for example, in U.S. Pat. No. 6,491,895 B2, US 2001/0041834 A1 or U.S. Pat. No. 6,696,040 B2.

The problem when using hyperpolarised gases in solution is the rapid depolarisation of these gases and the associated short half-lives that lie in the range of a few seconds. At the same time as the depolarisation, however, the property of the gas as an improved signal generator is also lost.

U.S. Pat. No. 6,488,910 B2 describes a method for the infiltration of hyperpolarised ³He and ¹²⁹Xe into a liquid by means of microbubbles. The liquid has a very low solubility for hyperpolarised ³He and ¹²⁹Xe. Transition of the hyperpolarised ³He and ¹²⁹Xe from the microbubble to the liquid is to be prevented here as far as possible, or at least suppressed, so that depolarisation is minimised and the relaxation time improved.

EP 0 968 000 B1 discloses contrast medium formulations that can be injected into the blood circulation for use in magnetic resonance-based analysis methods, and that consist of microvesicles that are filled with a mixture of hyperpolarised noble gas and an inert gas of high molecular weight. The presence of the inert gas here is intended to stabilise the hyperpolarised noble gas in such a way that the hyperpolarised gas is prevented from escaping through the vesicle wall and the hyperpolarised gas remains in the microvesicles.

According to the prior art, relatively complex methods are employed to try to bring gases with short-lived physical properties, in particular hyperpolarised gases, into a liquid in such a way that the gas remains in the gas phase in order to minimise the reduction of the half-life caused by the gas becoming dissolved in the liquid.

The object of the present invention is to provide a process by which gases with short-lived physical properties can be brought into solution in a simple and at the same time efficient manner.

This object is achieved by a process for dissolving a gas with short-lived physical properties in a liquid, comprising the steps of providing the gas with short-lived physical properties, providing the liquid and bringing the gas with short-lived physical properties into the liquid, said process being characterised in that the gas with short-lived physical properties is fed into the liquid via a semi-permeable membrane that is permeable to gas.

Surprisingly it has been discovered that the process according to the invention with semi-permeable membranes that are permeable to gas influences the half-life of the gases with short-lived physical properties significantly less than a process for direct solution of the gases in the liquid. With the process according to the invention, the half-life of the gas with short-lived physical properties remains at a high level. This applies in particular to hyperpolarised gases, as it has been discovered that semi-permeable membranes that are permeable to gas only slightly depolarise the hyperpolarised gases.

At the same time, with the use of membranes a direct molecular solution of the gases with short-lived physical properties in the liquid can be achieved. For the use of such solutions it is often very important that a stabile solution of the gases is obtained. Gas bubbles—even microscopically small bubbles—and host-guest inclusions are either in an unstable state or keep the gases trapped in molecules. Although the relaxation time of gases trapped in molecules is relatively long, the free diffusivity is lost, thus severely limiting their use as contrast media. Furthermore, the application of the gas-enriched liquid as a contrast medium using unstable solutions also entails the risk of a spontaneous formation of larger gas bubbles and hence the danger of vascular occlusions and embolisms, resulting in significant limitations in the application. In addition, the molecular solution also avoids the problem of foaming. The solutions can be quickly and reliably produced and are simple to use. Membranes allow the gas to be continuously dissolved in the liquid, so that larger volumes of gas are dissolved per unit of time than is the case with the injection of gas bubbles into the liquid. It is also important to ensure that all surfaces coming into contact with the gas or liquid are made of the cleanest possible inert materials with minimum differences in their magnetic susceptibility.

Within the context of the present invention, gases with short-lived physical properties are to be understood as gases whose specific physical property decays over time with half-lives of between a few milliseconds and several hours.

As already mentioned at the beginning, these include radioactive gases or hyperpolarised gases. Radioactive gases are employed, e.g. in positron emission tomography (PET). The radioactive gases contain so-called radioisotopes or radionuclides.

Hyperpolarised gases are employed for nuclear magnetic resonance spectroscopy (NMR) or nuclear spin tomography or magnetic resonance tomography (MRT). The use of hyperpolarised gases significantly improves the signal-to-noise ratio. The xenon isotopes ¹²⁹Xe or ¹³¹Xe or the helium isotope ³He are predominantly employed here.

In addition, the following isotopes are also to be considered relevant for gas compounds:

| | | | Gaseous |
|---|---|---|---|
| | | | compound |
| Isotope | Half-life | | (e.g.) |
| ¹⁰C | 19.5 | s | CO₂, CH₄, |
| ¹¹C | 20 | min | CFC* |
| ¹⁵C | 2.5 | s | |
| ¹³N | 10 | min | N₂, N₂O, NH₃ |
| ¹⁶N | 7 | s | |
| ¹⁷N | 4.2 | s | |
| ¹⁴O | 70 | s | O₂, CO₂ |
| ¹⁵O | 123 | s | |
| ¹⁹O | 29 | s | |
| ²⁰O | 14 | s | |
| ¹⁷F | 66 | s | SF₆, CFC* |
| ¹⁸F | 109 | min | |
| ²⁰F | 11 | s | |
| ²¹F | 4.3 | s | |
| ²²F | 4 | s | |
| ¹⁸Ne | 1.5 | s | Ne |
| ¹⁹Ne | 17 | s | |
| ²³Ne | 37 | s | |
| ²⁴Ne | 3.4 | min | |
| ³⁰S | 1.4 | s | SF₆ |
| ³¹S | 2.7 | s | |
| ³⁷S | 5 | min | |
| ³⁸S | 2.9 | h | |
| ³³Cl | 2.5 | s | CFC* |
| ³⁴Cl | 1.6 | s | |
| ^{34m}Cl | 32 | min | |
| ³⁸Cl | 37.3 | min | |
| ³⁹Cl | 55 | min | |
| ⁴⁰Cl | 1.4 | min | |
| ³⁵Ar | 1.8 | s | Ar |
| ⁴¹Ar | 1.8 | h | |
| ⁷⁴Kr | 20 | min | Kr |
| ⁷⁵Kr | 5.5 | min | |
| ⁷⁶Kr | 14.8 | h | |
| ⁷⁷Kr | 1.2 | h | |
| ^{79m}Kr | 55 | s | |
| ^{81m}Kr | 13 | s | |
| ^{83m}Kr | 1.9 | h | |
| ^{85m}Kr | 4.4 | h | |
| ⁸⁷Kr | 76 | min | |
| ⁸⁸Kr | 2.8 | h | Kr |
| ⁸⁹Kr | 3.2 | min | |
| ⁹⁰Kr | 33 | s | |
| ⁹¹Kr | 10 | s | |
| ⁹²Kr | 3 | s | |
| ⁹³Kr | 2 | s | |
| ⁹⁴Kr | 1.4 | s | |
| ¹¹⁸Xe | 6 | min | Xe |
| ¹¹⁹Xe | 6 | min | |
| ¹²⁰Xe | 40 | min | |
| ¹²¹Xe | 40 | min | |
| ¹²²Xe | 20 | h | |
| ¹²³Xe | 2 | h | |
| ¹²⁵Xe | 17 | h | |
| ^{125m}Xe | 60 | s | |
| ^{127m}Xe | 75 | s | |
| ¹³⁵Xe | 9.2 | h | |
| ^{135m}Xe | 15.6 | min | |
| ¹³⁷Xe | 4 | min | |
| ¹³⁸Xe | 17.5 | min | |
| ¹³⁹Xe | 43 | s | |
| ¹⁴⁰Xe | 16 | s | |
| ¹⁴¹Xe | 1.7 | s | |
| ²⁰²Rn | 13 | s | Rn |
| ²⁰³Rn | 45 | s | |
| ²⁰³mRn | 28 | s | |
| ²⁰⁴Rn | 75 | s | |
| ²⁰⁵Rn | 1.8 | min | |
| ²⁰⁶Rn | 6.5 | min | |
| ²⁰⁷Rn | 11 | min | |
| ²⁰⁸Rn | 23 | min | |
| ²⁰⁹Rn | 30 | min | |
| ²¹⁰Rn | 2.5 | h | |
| ²¹¹Rn | 15 | h | |
| ²¹²Rn | 25 | min | |
| ²¹⁹Rn | 4 | s | |
| ²²⁰Rn | 55 | s | |
| ²²¹Rn | 25 | min | |
| ²²³Rn | 43 | min | |
| ²²⁴Rn | 1.9 | h | |

Furthermore, molecular gases can also be made accessible by hydration of suitable molecules with double or triple bonds with para-¹H₂ or ortho-²H₂ (the so-called PHIP or PASADENA Experiment). The polarisation of the hydrogen isotopes can thereby also be transferred to other isotopes (e.g. ¹³C or ¹⁹F) using a nuclear spin in the low field (ALTADENA effect).

In a preferred embodiment, the process according to the invention is characterised in that the gas with short-lived physical properties is fed into the liquid bubble-free via the membrane. This results in a molecular solution of the gases with short-lived physical properties without foaming occurring in the solution. The process is therefore suitable for a wide variety of liquids, solutions and emulsions (amphiphilic substances, protein solutions, blood plasma, fatty emulsions, etc.) that in some cases tend to foam considerably. The liquids should thereby be as free as possible from paramagnetic and ferromagnetic impurities, as these would lead to rapid depolarisation of hyperpolarised gases. Attention must therefore be paid to the removal of dissolved O₂.

In a further preferred embodiment of the process according to the invention, the membrane is embedded in a housing in such a way that the housing is split by the membrane into at least one space for the liquid and at least one space for the gas, and that the space for the liquid has at least one inlet and one outlet and the space for the gas has at least one inlet for the gas via which the gas is admitted to the membrane. Particularly preferred is a continuous counterflow of gas and liquid by means of which the gas is enriched in the solution.

The membrane employed in the process according to the invention can be a flat membrane or at least one hollow fibre membrane. Hollow fibre membranes are preferably employed, with the hollow fibre membranes being arranged in a bundle.

Particularly advantageous are hollow fibre membranes having an outside diameter between 30 and 3000 μm, preferably between 50 and 500 μm. A favourable wall thickness of the hollow fibre membrane lies between 5 and 150 μm, particularly favourable being a thickness between 10 and 100 μm.

The membrane employed in the process according to the invention must on the one hand have a sufficient permeability for the gas with short-lived physical properties, and on the other hand remain impermeable over a sufficient period to the liquid into which the gas is fed.

The membrane employed in the process according to the invention can be hydrophilic or hydrophobic. In the event that a hydrophilic membrane is employed, this membrane preferably has a separating layer that is impermeable to the passage of liquid, but permeable to gas. The membrane employed in the process according to the invention is, however, preferably a hydrophobic membrane. In an advantageous embodiment, this hydrophobic membrane has a continuous microporous structure over its wall. Such membranes are described, for example, in DE 28 33 493 C3, DE 32 05 289 C2 or GB 2 051 665.

In a further advantageous embodiment the hydrophobic membrane has an integrally asymmetric structure with a microporous supporting layer and an impermeable or nanoporous separating layer adjoining this supporting layer. Such membranes with impermeable or nanoporous separating layer are described e.g. in EP 299 381 A1, WO 99/04891 or WO 00/43114.

The membranes for dissolving gas with short-lived physical properties in a liquid are preferably made of a polyolefin-based polymer. This base can be a single polyolefin or a mixture of several polyolefins.

Particularly preferred for dissolving gas with short-lived physical properties in a liquid are membranes made from polypropylene, polyethylene, polymethyl pentene and associated modifications, copolymers, blends or mixtures of these polyolefins together or with other polyolefins.

In addition, membranes consisting of silicone compounds or having a silicone coating are suitable for the process according to the invention. Furthermore, membranes made from a polymer based on an aromatic sulfone polymer, such as e.g. polysulfone, polyether sulfone or polyarylether sulfone, can also be considered suitable.

The process for dissolving gas with short-lived physical properties in a liquid is particularly suitable for dissolving hyperpolarised gases.

The process according to the invention is preferably characterised in that the hyperpolarised gas is xenon.

A wide range of liquids can be used for dissolving gas with short-lived physical properties. Examples of such liquids, in addition to those already mentioned, are lipofundin, blood plasma, glucose solution or halogenated blood substitutes (perfluorocarbons). The liquid in which the hyperpolarised gas is dissolved is preferably a physiological liquid.

In a particularly preferred process the liquid in which the hyperpolarised gas is dissolved is blood.

A solution of hyperpolarised gas in a liquid produced according to the invention can be used as a contrast medium for analytical methods based on magnetic resonance. The benefits of hyperpolarised gases as a contrast medium for analytical methods based on magnetic resonance are described extensively in U.S. Pat. No. 6,630,126 B2.

A solution of hyperpolarised gas in a liquid produced according to the invention can also be used for:
Xe-NMR or SPINOE-NMR in the field of biotechnology for investigating the structure of giant molecules or the dynamics of proteins
Surface-sensitive Xe-SPINOE or CP-NMR [9] in biological membranes or complex biomolecules on thin layers.
High and low-field MRT and functional MRT on living beings

Particularly in biophysical investigations for which water is frequently employed as the solution medium, only inadequate concentrations of hyperpolarised xenon in water have been achieved to date. With the process according to the invention it is possible to increase the concentration of hyperpolarised xenon in water or aqueous solutions to such an extent that it can also be used as a contrast medium for examinations in water. This is achieved in that the hyperpolarised xenon is led at elevated pressure, preferably through hollow fibre membranes. The hyperpolarised xenon is thereby also advantageously dissolved bubble-free.

The process according to the invention for dissolving hyperpolarised gas in a liquid is suitable both for imaging analysis methods, such as nuclear spin tomography or magnetic resonance tomography (MRT), and for spectroscopic analysis methods based on magnetic resonance, such as nuclear magnetic resonance spectroscopy (NMR).

The use of a solution of hyperpolarised gas in a liquid produced according to the invention as a contrast medium is particularly preferred for analytical methods based on magnetic resonance for examinations of human or animal organs and tissue.

The invention is described in greater detail by reference to the following examples, without limiting the field of application of the invention.

In the examples 1 and 2 described below, two arrangements were employed for performing the process according to the invention in which hyperpolarised xenon was continuously brought into a solution medium. The arrangements differ in the type of liquid pump. In arrangement 1, the liquid is pumped back and forth manually through the outer space surrounding the hollow fibre membrane of a hollow fibre membrane module and a connected reservoir by means of two syringes. The hollow fibre membrane module contained microporous polypropylene membranes such as those normally employed for the oxygenation of blood (Oxyphan PP50/280, Membrana GmbH). The hyperpolarised xenon was pumped through the lumina of the hollow fibre membranes of the membrane module and thereby diffused partially into the liquid. From here, the dissolved gas was transported further into a reservoir where it could be detected by means of an NMR measurement. In order to be able to compare the effectiveness of the method for different solution media, a hose with the xenon gas escaping from the membrane module was also routed along with the reservoir through the NMR coil. The NMR spectra can thus be referenced to the signal of the gaseous xenon.

In the second arrangement, the liquid was pumped in circulation through the test apparatus using a pneumatic diaphragm pump. As the pump employed contains no metallic or magnetic parts, it can also be operated in strong magnetic fields. This prototype thus enabled hyperpolarised xenon solution to be continuously produced. The gas volumetric flow in both arrangements was approx. 200 ml/min at a gas pressure above atmospheric of roughly 100 mbar. The liquid volumetric flow was set to approx. 240 ml/min.

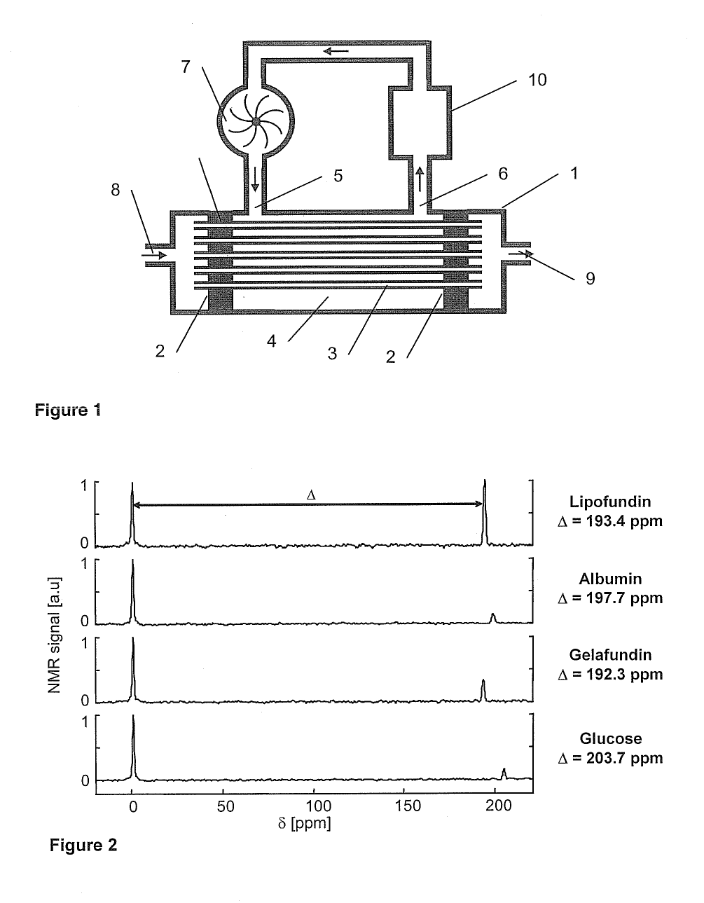

FIG. 1 shows schematically the second arrangement for dissolving hyperpolarised xenon in a liquid. The arrangement consists of a hollow fibre module 1 with hollow fibres 3 embedded in sealing compounds 2 at their ends. The liquid flows through the outer space 4 around the hollow fibres 3 via inlet port 5 and outlet port 6. In the arrangement shown in FIG. 1, the liquid is circulated by means of a pump 7. Alternatively, the liquid can also be pumped back and forth manually as described in the context of arrangement 1. On the lumen side, the hyperpolarised xenon is admitted to the hollow fibres 3 via inlet port 8 and outlet port 9. The hyperpolarised xenon dissolved in the liquid in detected in the NMR measuring cell 10.

### EXAMPLE 1

Various trials for bringing hyperpolarised xenon into various medically/biologically relevant liquids were performed using arrangement 1. The solution media used are medically safe and are already employed clinically. Lipofundin 20% is a fatty and albumin a protein-based nutritional solution for coma patients. Gelafundin is used as a plasma expander in cases of severe blood loss. Glucose solutions would be of particular interest as contrast media for imaging in brain examinations. In all the trials it was possible to dissolve the hyperpolarised xenon in the respective liquid via the membrane only in molecular form. In none of the solutions was xenon detected as a gas, i.e. in undissolved form. FIG. 2 shows the ¹²⁹Xe NMR spectra in the different solution media graphically. In order to reference the measured NMR spectra shown in FIG. 2 to the signal of the gaseous hyperpolarised xenon (chemical shift of 0 ppm), a further hose containing gaseous hyperpolarised xenon was routed through the measurement space as an external standard. The NMR signal of the dissolved xenon could be detected in each liquid at a chemical shift Δ characteristic of each liquid of between 192 and 204 ppm. It is consequently possible to bring xenon through the membrane into the different solution media without any loss of the spin polarisation, i.e. without measurable depolarisation. The differences in the intensities of the xenon signals in the dissolved state are attributable to the different concentrations of the substances and the individual solubility of xenon in these substances.

### EXAMPLE 2

Hyperpolarised xenon was brought into lipofundin via a hollow fibre membrane module using arrangement 2. FIG. 3 shows the signal strength of the hyperpolarised xenon dissolved in lipofundin as a function of the expired time after switching on the pump. The interrupted line in FIG. 3 marks the starting time of the pump. The second line marks the measured signal curve of the hyperpolarised xenon dissolved in lipofundin. A significant rise in the signal to approx. 75% of the maximum signal is already to be seen after only 2 seconds. The maximum value is reached after roughly 10 seconds. This shows clearly that hyperpolarised xenon brought into solution via semi-permeable, gas-permeable membranes is available almost immediately in the solution.

### EXAMPLES 3 AND 4, COMPARATIVE EXAMPLES 3A AND 4A

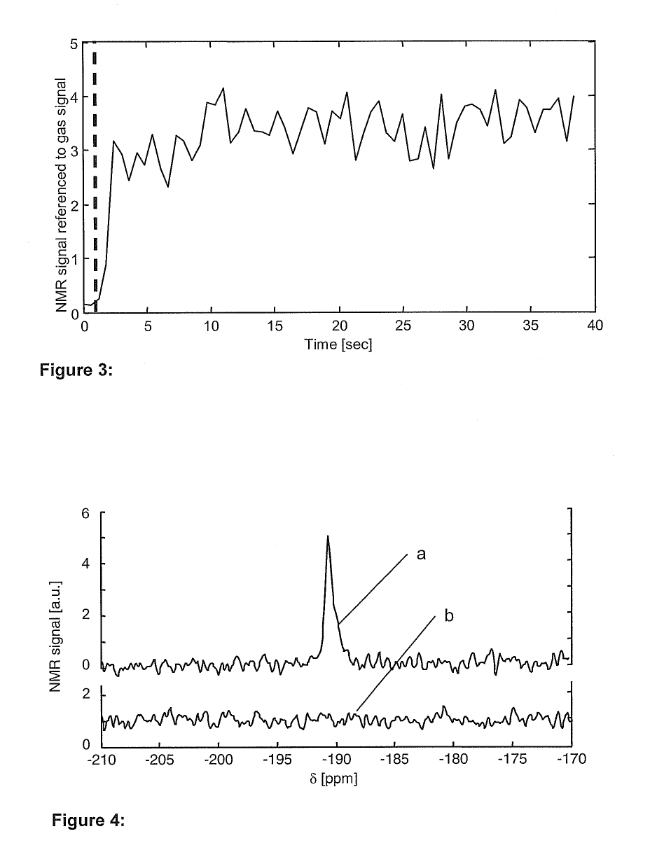

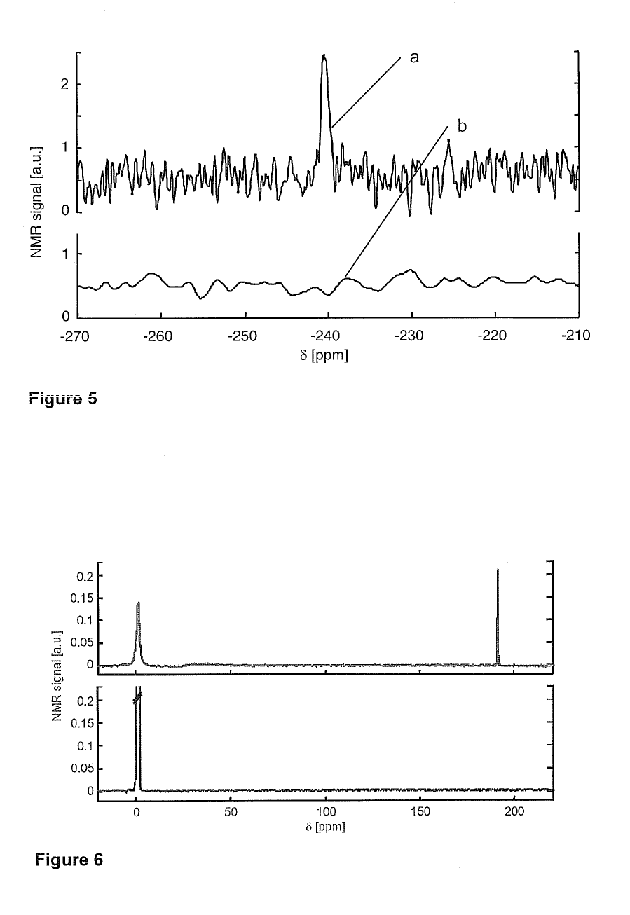

In examples 3 and 4, ¹²⁹Xe was brought into lipofundin (example 3) and DMSO (example 4) via a membrane by analogy with example 2. For comparison, these liquids were overlaid with ¹²⁹Xe and overflowed with ¹²⁹Xe. Simple overlaying or overflowing of the liquids with gas did not provide a measurable signal with either the liquid lipofundin (comparative example 3a) or with DMSO (comparative example 4a), whereas the inventive membrane-mediated process described was successful in both cases. Example 3 and comparative example 3a are shown in FIG. 4. The upper signal curve ‘a’ shows the ¹²⁹Xe signal in lipofundin for example 3 using the process of the present invention and the lower signal curve ‘b’ shows the ¹²⁹Xe signal in lipofundin for comparative example 3a, determined using the overlaying method. Example 4 and comparative example 4a are shown in FIG. 5. FIG. 5 shows a comparison of the ¹²⁹Xe signal dissolved in DMSO, whereby the upper signal curve ‘a’ (example 4) was determined using the process of the present invention and the lower signal curve ‘b’ (comparative example 4a) using the overlaying method. The signal curve ‘a’ shows in both cases a peak characteristic of dissolved hyperpolarised ¹²⁹Xe that could not be detected in signal curve ‘b’ in each case.

### EXAMPLE 5, COMPARATIVE EXAMPLE 5A

Hyperpolarised xenon was brought into water using a high-pressure hollow fibre module. The gas pressure of the hyperpolarised xenon flowing through the hollow fibres was 7 bar. The high-pressure hollow fibre module contained hollow fibre membranes of Celgard Type X50. These polypropylene-based hydrophobic membranes are typically employed for gassing and degassing of liquids. It was proved that hyperpolarised xenon can be dissolved in water in comparatively large quantities using the process according to the invention, despite the fact that the solubility coefficient of xenon in water has a very low value. FIG. 6 shows NMR spectra of hyperpolarised xenon in water, recorded using the process according to the invention at an elevated gas pressure of 7 bar (example 5), and of hyperpolarised xenon in water using the overlaying method, also at an elevated gas pressure of 7 bar (comparative example 5a). The control signal of the gaseous hyperpolarised xenon employed occurs at 0 ppm, and the signal of the hyperpolarised xenon dissolved in water occurs at roughly 190 ppm. The upper signal curve (example 5) was recorded using the process according to the invention. A strong signal of dissolved hyperpolarised xenon can be seen in this signal curve at 190 ppm. The lower curve (comparative example 5a) was recorded using the overlaying method. No dissolved hyperpolarised xenon was detected here. By means of example 5 and comparative example 5a it was possible to show that the efficiency of the process according to the invention in dissolving a gas with short-lived physical properties in a liquid can be further improved by increasing the gas pressure. This enables amphiphilic substances in aqueous solution to be well charged with xenon.

1. A process for dissolving a gas with short-lived physical properties in a liquid, comprising the steps of providing the gas with short-lived physical properties, providing the liquid and bringing the gas with short-lived physical properties into the liquid, said process being characterised in that the gas with short-lived physical properties is fed into the liquid via a semi-permeable membrane that is permeable to the gas.

2. The process according to claim 1, by bubble-free dissolving the gas into the liquid.

3. The process according to claim 1, wherein the membrane is embedded in a housing in such a way that the housing is split by the membrane into at least one space for the liquid and at least one space for the gas, and that the space for the liquid has at least one inlet and one outlet and the space for the gas has at least one inlet for the gas via which the gas is admitted to the membrane.

4. The process according to claim 1, wherein the membrane is at least one hollow fibre membrane.

5. The process according to one or more of claims 1 to 4, wherein the membrane is hydrophobic.

6. The process according to claim 5, wherein the membrane has a microporous structure.

7. The process according to claim 6, wherein the membrane has an impermeable or nanoporous separating layer.

8. The process according to claim 1, wherein the membrane is made from a polymer based on at least one polyolefin.

9. The process according to claim 8, wherein at least one polyolefin is polypropylene or polymethyl pentene.

10. The process according to claim 1, wherein the membrane is a silicone membrane or has a silicone coating.

11. The process according to claim 1, wherein the membrane is made from a polymer based on an automatic sulfone polymer.

12. The process according to claim 1, wherein the gas with short-lived physical properties is a hyperpolarised gas.

13. The process according to claim 12, wherein the hyperpolarised gas is xenon.

14. The process according to claim 13, wherein the liquid in which the hyperpolarised gas is dissolved in a physiological liquid.

15. The process according to claim 14, wherein the physiological liquid is blood plasma.

16. (canceled)

17. A contrast medium for analytical methods comprising a solution of hyperpolarised gas in a liquid, wherein the analytical methods being based on magnetic resonance.

18. A contrast medium for analytical methods comprising a solution of hyperpolarised gas in a liquid, wherein the analytical methods being based on magnetic resonance for examination of human or animal organs and tissue.

19. The contrast medium according to claim 17, wherein said solution being produced by the process of claim 1.

20. The contrast medium according to claim 18, wherein said solution being produced by the process of claim 1.
